(19) United States
(12) Patent Application Publication
(10) Pub. No.: US 2021/0220367 A1
(43) Pub. Date: Jul. 22, 2021

(54) INHALABLE FORMULATION OF A SOLUTION CONTAINING GLYCOPYRROLATE AND OLODATEROL HYDROCHLORIDE

(71) Applicants: Cai Gu Huang, Shrewsbury, MA (US); Xiao Qian Wang, Shanghai (CN); Ning He, Shanghai (CN)

(72) Inventors: Cai Gu Huang, Shrewsbury, MA (US); Xiao Qian Wang, Shanghai (CN); Ning He, Shanghai (CN)

(73) Assignee: Cai Gu Huang, Shrewsbury, MA (US)

(21) Appl. No.: 17/152,049

(22) Filed: Jan. 19, 2021

Related U.S. Application Data
(60) Provisional application No. 62/963,525, filed on Jan. 20, 2020.

(51) Int. Cl. A61K 31/538; A61K 31/40; A61K 47/12; A61K 47/18; A61K 9/00; A61K 47/02

(52) U.S. Cl. CPC A61K 31/538; A61K 31/40; A61K 47/12; A61K 47/02; A61K 47/183; A61K 9/0078; A61K 47/186

(57) ABSTRACT

The present invention relates to a liquid pharmaceutical preparation and a method for administering the pharmaceutical preparation by nebulizing the pharmaceutical preparation in an inhaler. The propellant-free pharmaceutical preparation comprises: (a) glycopyrrolate or a salt thereof (b) olodaterol or a salt thereof; (b) a solvent; (c) a pharmacologically acceptable preservative, and (d) a pharmacologically acceptable stabilizer, and optionally other pharmacologically acceptable additives.

### PRIORITY STATEMENT

This application claims the benefit of the filing date of U.S. Provisional patent Application No. 62/963,525, filed on Jan. 20, 2020, the contents of which are incorporated herein by reference in its entirety.

### BACKGROUND OF THE INVENTION

Glycopyrronium bromide (also known as glycopyrrolate), chemically known as (3RS)-3-[(2SR)-(2-cyclopentyl-2-hydroxy-2-penylacetyl)oxy]-1,1-dimethlypyrrolidinium bromide, has the following chemical structure:

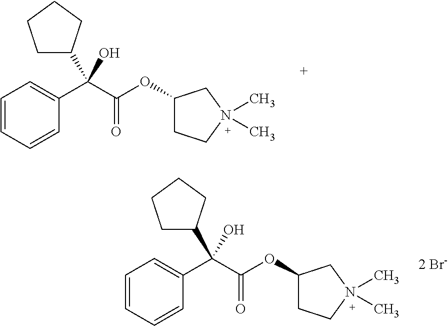

Olodaterol hydrochloride, chemically known as 2H-1,4-Benzoxazin-3H(4H)-one, 6-hydroxy-8-[(1R)-1-hydroxy-2-[[2-(4-methoxyphenyl)-1,1-dimethylethyl]-amino] ethyl], monohydrochloride, is disclosed in U.S. Pat. Nos. 7,220,742, 7,491,719, 7,056,916, and 7,727,984, and has the following chemical structure:

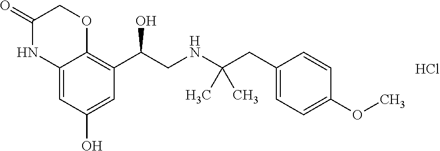 [SMILES: COc1ccc(CC(C)(C)NC[C@H](O)c2cc(O)cc3c2OCC(=O)N3)cc1 ; Cl]

Glycopyrrolate is a long-acting muscarinic antagonist, which is often referred to as an anticholinergic, approved for the long-term maintenance treatment of airflow obstruction in patients with chronic obstructive pulmonary disease (COPD), including chronic bronchitis and/or emphysema. In chronic obstructive pulmonary disease, acetylcholine is released to airway smooth muscle and acts reversibly through postsynaptic muscarinic receptors to mediate airway smooth muscle contraction and mucus secretion. Inhaled anticholinergic agents can block muscarinic receptors on airway smooth muscle to inhibit bronchoconstriction.

Olodaterol is a long-acting beta-2-adrenergic agonist (LABA) that activates beta-2 adrenoreceptors on airway smooth muscle, causing bronchodilation. Beta-2 receptors are the adrenergic receptors in bronchial smooth muscle.

These two compounds have valuable pharmacological properties. Glycopyrronium and olodaterol can provide therapeutic benefit in the treatment of asthma or chronic obstructive pulmonary disease, including chronic bronchitis and emphysema.

The present invention relates to propellant-free inhalable formulations of glycopyrronium and olodaterol or their pharmaceutically acceptable salts dissolved in water, in conjunction with inactive ingredients including pH-adjusting ingredients, which may be administered using a soft mist or nebulization inhalation device, and the propellant-free inhalable aerosols resulting therefrom. The pharmaceutical formulations disclosed in the current invention are especially suitable for soft mist inhalation or nebulization inhalation, which have much better lung depositions (typically up to 55-60%), compared to dry powder inhalation methods.

The pharmaceutical formulations of the present invention are particularly suitable for administering the active substances by soft mist or nebulization inhalation, and are especially useful for treating asthma and chronic obstructive pulmonary disease.

### SUMMARY OF THE INVENTION

The present invention relates to pharmaceutical formulations of glycopyrronium and olodaterol or their pharmaceutically acceptable salts or solvates which can be administered by soft mist or nebulization inhalation. The pharmaceutical formulations according to the invention meet high quality standards.

One aspect of the present invention is to provide an aqueous pharmaceutical formulation containing glycopyrronium and olodaterol or their pharmaceutically acceptable salts or solvates, which meets the high standards needed to achieve optimum nebulization of the formulation using the inhalers mentioned hereinbefore. The stability of the active substances in the formulation should be a storage time of some years, preferably at least one year, more preferably at least three years.

Another aspect is to provide a propellant-free formulation of a solution containing glycopyrronium and olodaterol or their pharmaceutically acceptable salts or solvates, which is nebulized under pressure using an inhaler device, which is preferably a soft mist or nebulization inhaler device, the aerosol delivered by the resulting nebulization falling reproducibly within a specified range for particle size.

More specifically, another aspect is to provide a stable pharmaceutical formulation of an aqueous solution containing glycopyrronium and olodaterol or their pharmaceutically acceptable salts or solvates and other excipients which can be administered by soft mist or nebulization inhalation.

### BRIEF DESCRIPTION OF THE DRAWINGS

FIG. 1 shows a longitudinal section through the atomizer in the stressed state;

FIG. 2 shows a counter element of the atomizer;

FIG. 3 shows the particle size distribution of droplets when sample IV is sprayed by a soft mist inhaler as described in example 3.

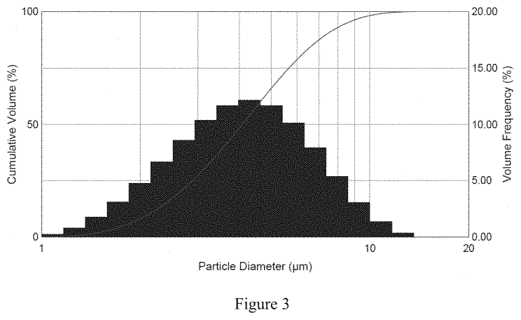

FIG. 4 shows the aerodynamic particle size distribution determined using a Next Generation Pharmaceutical Impactor (NGI) as described in Example 5.

### DETAILED DESCRIPTION OF THE INVENTION

It is advantageous to administer a liquid formulation of an active substance(s) without propellant gases using suitable inhalers, in order to achieve a better distribution of the active substance(s) in the lung. Furthermore, it is important to maximize the lung deposition of the drug delivered by inhalation.

A traditional pMDI (pressurized metered dose inhaler) or DPI (dry powder inhalaler) can only delivery about 20-30% of a drug into the lung, resulting in a significant amount of drug being deposited on the mouth and throat, which can then go to the stomach and cause unwanted side effects and or secondary absorption through oral digest system.

Therefore, there is a need to significantly increase lung deposition when administering a drug by inhalation. The soft mist or nebulization inhalation device disclosed in US2019/0030268 can significantly increase the lung deposition of inhalable drugs.

These inhalers nebulize a small amount of a liquid formulation within a few seconds into an aerosol that is suitable for therapeutic inhalation. The inhalers are particularly suitable for use with the liquid formulations disclosed herein.

Soft mist or nebulization devices suitable for use with the aqueous pharmaceutical formulations of the present invention are those in which an amount of less than about 70 microliters of pharmaceutical solution can be nebulized in one puff, such as less than about 30 microliters, or such as less than about 15 microliters, so that the inhalable part of the aerosol corresponds to a therapeutically effective quantity. in an embodiment, the average particle size of an aerosol formed from one puff is less than about 15 microns, such as less than about 10 microns.

A device of this kind for the propellant-free administration of a metered amount of a liquid pharmaceutical composition for inhalation is described in detail, for example, in US2019/0030268 entitled “inhalation atomizer comprising a blocking function and a counter”.

The pharmaceutical formulation in the nebulizer is converted into an aerosol destined for the lungs. The nebulizer uses high pressure to spray the pharmaceutical formulation.

The pharmaceutical formulation of the invention is stored in a reservoir in these kind of inhalers. The pharmaceutical formulation must not contain any ingredients which might interact with the inhaler and affect the pharmaceutical quality of the solution or of the aerosol produced. In addition, it is desirable that the active substances in pharmaceutical formulations are stable when stored and are capable of being administered directly.

The pharmaceutical formulations of the current invention for the inhaler described above preferably contain additives, such as the disodium salt of edetic acid (disodium edetate), to reduce the incidence of spray anomalies and to stabilize the solutions. The pharmaceutical formulations of the invention preferably have a minimum concentration of disodium edetate.

Therefore, one aspect of the present invention is to provide an aqueous pharmaceutical formulation containing glycopyrronium and olodaterol or their pharmaceutically acceptable salts, which meets the high standards needed to achieve optimum nebulization of the solution using the inhalers mentioned hereinbefore. In an embodiment, the active substances in the pharmaceutical formulation are stable, and have a storage time of some years, for example about one year, or about three years.

Another aspect of the current invention is to provide propellant-free formulations of solutions containing glycopyrronium and olodaterol or their pharmaceutically acceptable salts which are nebulized under pressure using an inhaler, such as a soft mist inhaler or other nebulization inhaler, wherein the resulting aerosol produced by the inhaler falls reproducibly within a specified range for particle size.

Another aspect is to provide an aqueous pharmaceutical formulation as a solution containing glycopyrronium and olodaterol or their pharmaceutically acceptable salts, and other inactive excipients which can be administered by inhalation.

According to the invention, any pharmaceutically acceptable salts or solvates of glycopyrronium and olodaterol may be used in the formulations. In one aspect of the invention, the pharmaceutically acceptable salt or solvate of glycopyrronium is glycopyrronium bromide and the pharmaceutically acceptable salt or solvate of olodaterol is olodaterol hydrochloride.

Within the scope of the present invention glycopyrrolate and olodaterol hydrochloride are preferred.

In the formulations according to the invention, the active substances are preferably selected from combinations of glycopyrrolate and olodaterol hydrochloride.

In the formulations according to the invention, glycopyrronium and olodaterol or their pharmaceutically acceptable salts are dissolved in a solvent. In an embodiment, the solvent is water.

The concentration of the glycopyrronium and olodaterol or their pharmaceutically acceptable salts in the finished pharmaceutical preparation depends on the therapeutic effects. The concentration of glycopyrrolate in the formulation is between about 10 mg/100 ml and about 6 g/100 ml, preferably between about 100 mg/100 ml and about 500 mg/100 ml. The concentration of olodaterol is between about 2 mg/100 ml and about 1.5 g/100 ml, preferably between about 0.2 mg/100 ml and about 230 mg/100 ml.

In an embodiment, a therapeutically effective dose of glycopyrrolate is from about 50 mg/100 ml to about 300 mg/100 ml and a therapeutically effective dose of olodaterol is from about 10 mg/100 ml to about 50 mg/100 ml.

In one aspect of the formulations according to the invention, the formulation includes an acid and/or a base, as pH adjusting agent. In an embodiment, the pH adjusting agent is citric acid and/or a salt thereof.

Other comparable pH adjusting agents can be used in the present invention. Other pH adjusting agents include, but are not limited to, hydrochloric acid and sodium hydroxide.

Adjusting the pH can advantageously provide better stability of the active substances. Typically, the pH ranges from about 2.0 to about 7.5. In an embodiment, the pH ranges from about 3.0 to about 5.0. In an embodiment, the pH ranges from about 3.0 to about 4.0.

In one embodiment, the formulations include edetic acid (EDTA) or a salt thereof, such as disodium edetate or edetate disodium dihydrate, as a stabilizer or complexing agent. In an embodiment, the formulation contains edetic acid and/or salt thereof.

Other comparable stabilizers or complexing agents can be used in the present invention. Examples of stabilizers or complexing agents include, but are not limited to, citric acid monohydrate, anhydrous citric acid, edetate disodium, edetate disodium dihydrate.

A complexing agent is a molecule which is capable of entering into complex bonds. Preferably, these compounds have the effect of complexing cations. The concentration of the stabilizers or complexing agents is typically about 1 mg/100 ml to about 500 mg/100 ml. In an embodiment, the concentration of the stabilizers or complexing agents is about 10 mg/100 ml to about 200 mg/100 ml. In one embodiment, the stabilizer is edetate disodium dihydrate in a concentration of about 1 mg/100 ml to about 500 mg/100 ml.

In an embodiment the glycopyrrolate and olodaterol hydrochloride are present in solution.

In another embodiment, all the ingredients of the formulation are present in solution.

The formulations may further comprise additives. The term “additives,” as used herein, means any pharmacologically acceptable and therapeutically useful substance which is not an active substance, but can be formulated together with the active substances, in order to improve the qualities of the formulation. Preferably, these substances have no appreciable pharmacological effects or at least no undesirable pharmacological effect in the context of the desired therapy.

Additives include, but are not limited to, for example, other stabilizers, complexing agents, antioxidants, surfactants, and/or preservatives which prolong the shelf life of the finished pharmaceutical formulation, vitamins and/or other additives known in the art.

In one aspect of the invention, the formulation further comprises a suitable preservative to protect the formulation from contamination with pathogenic bacteria. In one embodiment, the preservative comprises benzalkonium chloride, benzoic acid, or sodium benzoate. Preferred formulations contain only benzalkonium chloride. The amount of preservative ranges from about 2 mg/100 ml to about 1000 mg/100 ml. In a preferred embodiment, the preservative is benzalkonium chloride in an amount of about 20 mg/100 ml to about 50 mg/100 ml.

To produce the propellant-free aerosols according to the invention, the pharmaceutical formulations containing glycopyrronium and olodaterol or their pharmaceutically acceptable salts are preferably used with an inhaler of the kind described hereinbefore.

The inhaler disclosed in US2019/0030268 entitled “inhalation atomizer comprising a blocking function and a counter” is an example of an inhaler that is suitable for use with the formulations of the present invention.

The pharmaceutical formulation is converted by the nebulizer into aerosol destined for the lungs. The nebulizer uses high pressure to spray the formulation.

The inhalable device can be carried anywhere by the patient, since it has a cylindrical shape and handy size of less than about 8 cm to about 18 cm long, and about 2.5 cm to about 5 cm wide. The nebulizer sprays out a defined volume of the pharmaceutical formulation through small nozzles at high pressures, so as to produce an inhalable aerosol.

The preferred atomizer comprises an atomizer 1, a fluid 2, a vessel 3, a fluid compartment 4, a pressure generator 5, a holder 6, a drive spring 7, a delivering tube 9, a non-return valve 10, a pressure room 11, a nozzle 12, a mouthpiece 13, an aerosol 14, an air inlet 15, an upper shell 16, and an inside part 17.

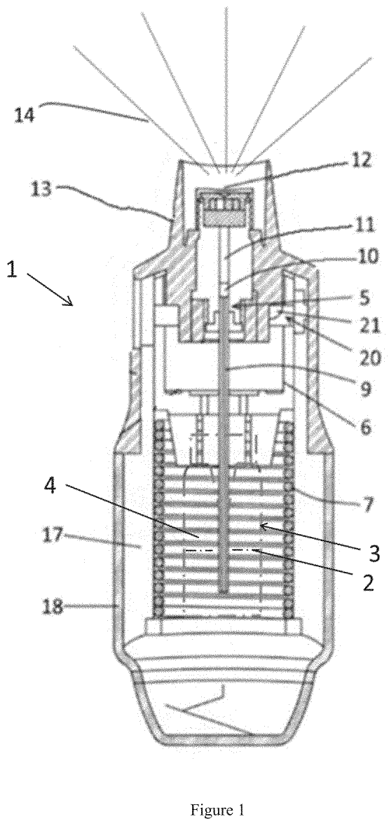

The inhalation atomizer 1 comprising the block function and the counter described above for spraying a medicament fluid 2 is depicted in the FIG. 1 in a stressed state. The atomizer 1 comprising the block function and the counter described above is preferably a portable inhaler and requires no propellant gas.

FIG. 1 shows a longitudinal section through the atomizer in the stressed state.

For the typical atomizer 1 comprising the block function and the counter described above, an aerosol 14 that can be inhaled by a patient is generated through atomization of the fluid 2, which is preferably formulated as a medicament liquid. The medicament is typically administered at least once a day, more specifically multiple times a day, preferably at predestined time gaps, according to how seriously the illness affects the patient.

In an embodiment, the atomizer 1 described above has a substitutable and insertable vessel 3, which contains the medicament fluid 2. Therefore, a reservoir for holding the fluid 2 is formed in the vessel 3. Specifically, the medicament fluid 2 is located in the fluid compartment 4 formed by a collapsible bag in the vessel 3.

In an embodiment, the amount of fluid 2 for the inhalation atomizer 1 described above is in the vessel 3 to provide, for example, up to 200 doses. A classical vessel 3 has a volume of about 2 to about 10 ml. A pressure generator 5 in the atomizer 1 is used to deliver and atomize the fluid 2, in a predetermined dosage amount. Therefore, the fluid 2 can be released and sprayed in individual doses, specifically doses of from about 5 to about 30 microliters.

In one embodiment, atomizer 1 described above may have a pressure generator 5, a holder 6, a drive spring 7, a delivering tube 9, a non-return valve 10, a pressure room 11, and a nozzle 12 in the area of a mouthpiece 13. The vessel 3 is latched by the holder 6 in the atomizer 1 so that the delivering tube 9 is plunged into the vessel 3. The vessel 3 can be separated from the atomizer 1 for substitution.

In an embodiment, when drive spring 7 is stressed in an axial direction, the delivering tube 9 and the vessel 3 along with the holder 6 will be shifted downwards. Then the fluid 2 will be sucked into the pressure room 11 through delivering tube 9 and the non-return valve 10.

In an embodiment, after releasing the holder 6, the stress is eased. During this process, the delivering tube 9 and closed non-return valve 10 are shifted upward by releasing the drive spring 7. Consequently, the fluid 2 is under pressure in the pressure room 11. The fluid 2 is then pushed through the nozzle 12 and atomized into an aerosol 14 by the resulting pressure. A patient can inhale the aerosol 14 through the mouthpiece 13, while the air is sucked into the mouthpiece 13 through air inlets 15.

In an embodiment, the inhalation atomizer 1 described above has an upper shell 16 and an inside part 17, which can be rotated relative to the upper shell 16. A lower shell 18 is manually operable to attach onto the inside part 17. The lower shell 18 can be separated from the atomizer 1 so that the vessel 3 can be substituted and inserted.

In an embodiment, the inhalation atomizer 1 described above may have a lower shell 18, which carries the inside part 17, and is rotatable relative to the upper shell 16. As a result of rotation and engagement between the upper unit 17 and the holder 6, through a gear 20, the holder 6 axially moves the counter in response to the force of the drive spring 7 when the drive spring 7 is stressed.

In an embodiment, in the stressed state, the vessel 3 is shifted downwards until it reaches a final position, which is demonstrated in FIG. 1. The drive spring 7 is stressed in this final position. Then the holder 6 is clasped. Therefore, the vessel 3 and the delivering tube 9 are prevented from moving upwards so that the drive spring 7 is stopped from easing.

In an embodiment, the atomizing process occurs after the holder 6 is released. The vessel 3, the delivering tube 9 and the holder 6 are shifted back by the drive spring 7 to the beginning position. This is referred to herein as major shifting. When the major shifting occurs, the non-return valve 10 is closed and the fluid 2 is under the pressure in the pressure room 11, and then the fluid 2 is pushed out through the delivering tube 9 and atomized by the pressure.

In an embodiment, the inhalation atomizer 1 described above may have a clamping function. During the clamping, the vessel 3 preferably performs a lifting shift for the withdrawal of the fluid 2 during the atomizing process. The gear 20 has sliding surfaces 21 on the upper shell 16 and/or on the holder 6, which allows the holder 6 to move axially when the holder 6 is rotated relative to the upper shell 16.

In an embodiment, the holder 6 is not blocked for too long and can carry on the major shifting. Therefore, the fluid 2 is pushed out and atomized.

In an embodiment, when the holder 6 is in the clamping position, the sliding surfaces 21 move out of engagement. Then the gear 20 releases the holder 6 for the opposite axial shift.

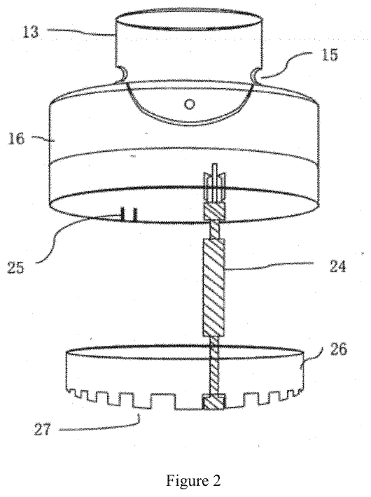

In an embodiment, the atomizer 1 includes a counter element shown in FIG. 2. The counter element has a worm 24 and a counter ring 26. The counter ring 26 is preferably circular and has dentate part at the bottom. The worm 24 has upper and lower end gears. The upper end gear contacts with the upper shell 16. The upper shell 16 has inside bulge 25. When the atomizer 1 is employed, the upper shell 16 rotates, and when the bulge 25 passes through the upper end gear of the worm 24, the worm 24 is driven to rotate. The rotation of the worm 24 drives the rotation of the counter ring 26 through the lower end gear so as to result in a counting effect.

In an embodiment, the locking mechanism is realized mainly by two protrusions. Protrusion A is located on the outer wall of the lower unit of the inside part. Protrusion B is located on the inner wall of the counter. The lower unit of the inside part is nested in the counter. The counter can rotate relative to the lower unit of the inside part. Because of the rotation of the counter, the number displayed on the counter can change as the actuation number increases, and can be observed by the patient. After each actuation, the number displayed on the counter changes. Once the predetermined number of actuations is achieved, Protrusion A and Protrusion B will encounter each other and the counter will be prevented from further rotation. This blocks the atomizer, stopping it from further use. The number of actuations of the device can be counted by the counter.

The nebulizer described above is suitable for nebulizing the aerosol preparations according to the invention to form an aerosol suitable for inhalation. Nevertheless, the formulation according to the invention can also be nebulized using other inhalers apart from those described above, such as an ultrasonic vibrating mesh nebulizer or a compressed air nebulizer.

### EXAMPLES

Materials and Reagents:

50% benzalkonium chloride aqueous solution purchased from Merck,

Edetate disodium dihydrate purchased from Merck,

Citric acid purchased from Merck,

Sodium hydroxide purchased from Titan reagents,

Hydrochloric acid purchased from Titan reagents,

Glycopyrrolate is commercially available and may be purchased from Nanjing Daqin Pharma Co., Ltd.,

Olodaterol is commercially available and may be purchased from Shanghai Caerulum Pharma Discovery Co., Ltd.

### Example 1

Sample I, sample II, and sample III inhalation solutions were prepared as follows: 50% benzalkonium chloride aqueous solution and Edetate Disodium Dihydrate according to the amounts provided in table 1 and table 2, were dissolved in 90 ml of purified water, and the resulting solution adjusted to target pH with citric acid, hydrochloric acid, or sodium hydroxide. Glycopyrrolate and olodaterol hydrochloride according to the amounts provided in table 1 and table 2 were added to the solution, and the resulting mixture was sonicated until completely dissolved. Finally, purified water was added to provide a final volume of 100 ml.

TABLE 1
Ingredient contents of sample I of 100
ml inhalation solution formulation

| Ingredients | Sample I | |
|---|---|---|
| Glycopyrrolate | 11.3 | mg |
| Olodaterol hydrochloride | 2.5 | mg |
| Edetate Disodium Dihydrate | 1 | mg |
| 50% benzalkonium chloride aqueous | 2 | mg |
| solution | | |

| | |
|---|---|
| Hydrochloric acid | To pH 2.0 |
| Purified water | Added to 100 ml |

TABLE 2
Ingredient contents of sample II and sample
III of 100 ml inhalable formulation

| Ingredients | Sample II | | Sample III | |
|---|---|---|---|---|
| Glycopyrrolate | 5.65 | g | 5.65 | g |
| Olodaterol hydrochloride | 1.25 | g | 1.25 | g |
| Edetate Disodium Dihydrate | 0.5 | g | 0.5 | g |
| 50% benzalkonium chloride | 1 | g | 1 | g |
| aqueous solution | | | | |

| | | |
|---|---|---|
| Citric acid or sodium hydroxide | To pH 4.0 | To pH 6.0 |
| Purified water | Added to 100 ml | Added to 100 ml |

### Example 2

Sample IV inhalation solution was prepared as follows: 50% benzalkonium chloride aqueous solution and Edetate Disodium Dihydrate according to the amounts provided in table 4, were dissolved in 90 ml purified water, and the resulting solution adjusted to the target pH with citric acid, hydrochloric acid, or sodium hydroxide. Glycopyrrolate and olodaterol hydrochloride according to the amounts provided in table 4 were added to the solution, and the resulting solution was sonicated until completely dissolved. Finally, purified water was added to a final volume of 100 ml.

TABLE 4
Ingredient contents of IV of 100 ml inhalable formulation

| Ingredients | Sample IV | |
|---|---|---|
| Glycopyrrolate | 113 | mg |
| Olodaterol hydrochloride | 25 | mg |
| Edetate Disodium Dihydrate | 10 | mg |
| 50% benzalkonium chloride aqueous | 20 | mg |
| solution | | |

| | |
|---|---|
| Citric acid or sodium hydroxide | To pH 4.0 |
| Purified water | Added to 100 ml |

### Example 3

Sample IV was sprayed using a soft mist inhalation device. A Malvern Spraytec (STP5311) instrument was used to measure the particle size of the droplets. The results are shown in table 5.

TABLE 5
Particle size distribution of sample IV by using soft mist inhalation

| | |
|---|---|
| Sample Number | Droplet size (μm) |

| | | |
|---|---|---|
| Sample IV | D10 | 2.08 |
| | D50 | 3.91 |
| | D90 | 6.93 |

### Example 4

Influence of Different pH on Stability:

The stability of glycopyrrolate (referred to as GB) and olodaterol hydrochloride (referred to as OH) solution is highly dependent on the pH. Seven samples were prepared, having pH 2.5, 3.0, 3.5, 4.0, 4.5, 5.0, and unadjusted pH. 50% benzalkonium chloride aqueous solution and Edetate Disodium Dihydrate according to the amounts in table 6 were dissolved in 190 ml of purified water, and the resulting solution was adjusted to the target pH with citric acid, hydrochloric acid, or sodium hydroxide. Glycopyrrolate and olodaterol hydrochloride according to the amounts provided in table 6 were added to the solution, and the resulting mixture was sonicated until completely dissolved. Finally, purified water was added to provide a final volume of 200 ml.

The formulae of the 7 samples are shown in Table 6. The preparation method is the same as Example 1. The experimental results of influencing factors are shown in Table 7.

TABLE 6
Formulation design of GB and OH compound screening at different pH values

| Ingredients | sample 1 | | sample 2 | | sample 3 | | sample 4 | | sample 5 | | sample 6 | | sample 7 | |
|---|---|---|---|---|---|---|---|---|---|---|---|---|---|---|
| Glycopyrrolate | 33.9 | mg | 33.9 | mg | 33.9 | mg | 33.9 | mg | 33.9 | mg | 33.9 | mg | 33.9 | mg |
| Olodaterol | 7.5 | mg | 7.5 | mg | 7.5 | mg | 7.5 | mg | 7.5 | mg | 7.5 | mg | 7.5 | mg |
| hydrochloride | | | | | | | | | | | | | | |
| Edetate Disodium | 3 | mg | 3 | mg | 3 | mg | 3 | mg | 3 | mg | 3 | mg | 3 | mg |
| Dihydrate | | | | | | | | | | | | | | |
| 50% benzalkonium | 6 | mg | 6 | mg | 6 | mg | 6 | mg | 6 | mg | 6 | mg | 6 | mg |
| chloride aqueous | | | | | | | | | | | | | | |
| solution | | | | | | | | | | | | | | |
| Anhydrous citric | Adjust to | | Adjust to | | Adjust to | | Adjust to | | Adjust to | | Adjust to | | pH not | |
| acid or sodium | pH 2.5 | | pH 3.0 | | pH 3.5 | | pH 4.0 | | pH 4.5 | | pH 5.0 | | adjusted | |
| hydroxide | | | | | | | | | | | | | | |
| Purified water | 30 | ml | 30 | ml | 30 | ml | 30 | ml | 30 | ml | 30 | ml | 30 | ml |

TABLE 7
Stability at different pH values

| | | conditions | | | | | | |
|---|---|---|---|---|---|---|---|---|
| | | | | | | | | pH not |
| | | pH 2.5 | pH 3.0 | pH 3.5 | pH 4.0 | pH 4.5 | pH 5.0 | adjusted |
| | | sample | | | | | | |
| | | Sample 1 | Sample 2 | Sample 3 | Sample 4 | Sample 5 | Sample 6 | Sample 7 |
| | pH | 2.54 | 3.08 | 3.61 | 4.06 | 4.63 | 5.18 | 5.86 |
| 0 Day | GB-J (%) | 0.049 | — | — | — | 3.652 | 3.765 | 4.482 |
| 60° C. | GB-J (%) | 0.29 | 0.154 | 0.222 | 3.06 | 5.70 | 6.431 | 6.183 |
| 7 Days | | | | | | | | |
| 60° C. | GB-J (%) | 0.505 | 0.266 | 0.267 | 4.03 | 6.67 | 6.7 | 7.161 |
| 14 Days | | | | | | | | |

GB-J is a major degradation product of glycopyrrolate. The chemical name of GB-J is 2-cyclopenty-2-hydroxy-2-phenylacetic acid.

The above studies confirmed that the stability of a glycopyrrolate and olodaterol hydrochloride solution is highly dependent on the formulation pH. As can be seen from Table 7, the GB and OH formulations are stable at pH 2.5 to 6.0, with the highest stability at pH 3.0-4.0.

### Example 5

Aerodynamic Particle Size Distribution:

The aerodynamic particle size distribution was determined using a Next Generation Pharmaceutical Impactor (NGI). The soft mist inhaler was held close to the NGI inlet until no aerosol was visible. The flow rate of the NGI was set to 30 L/minute and was operated under ambient temperature and a relative humidity (RH) of 90%.

The solution of sample 2 was discharged into the NGI. Fractions of the dose were deposited at different stages of the NGI, in accordance with the particle size of the fraction. Each fraction was washed from the stage and analyzed using HPLC.

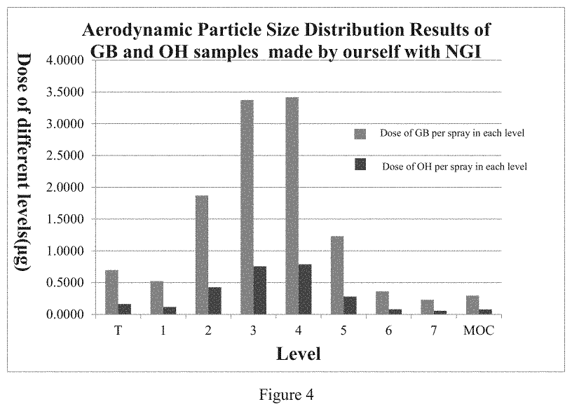

The particle size distribution was expressed in terms of mass median aerodynamic diameter (MMAD) and Geometric Standard Deviation (GSD). The results showed that the MMAD values of both glycopyrronium bromide and formoterol fumarate were less than 6 μm, and the GSD values of both glycopyrronium bromide and formoterol fumarate were less than 2 μm. The sample is sample 2 as described in Example 4 (GB 1.13 mg/ml; pH 3.0). The results are provided in Table 8 below. The aerodynamic particle size distribution of sample 2 was investigated by means of the original research device of TB-OH and a new generation of cascade impactor. The result is shown in FIG. 4.

TABLE 8
Aerodynamic particle size distribution

| Particle size parameter | Glycopyrrolate | Olodaterol hydrochloride |
|---|---|---|
| MMAD (μm) | 4.05 | 4.01 |
| GSD | 1.77 | 1.79 |

### Example 6

Stability Experiment:

Sample V-VII inhalation solution was prepared as follows:

50% benzalkonium chloride aqueous solution and Edetate Disodium Dihydrate according to the amounts provided in table 9 were dissolved in 190 ml purified water, and the resulting solution was adjusted to the target pH with citric acid, hydrochloric acid, or sodium hydroxide. Glycopyrrolate and olodaterol hydrochloride according to the amounts provided in table 9 were added to the solution, and the resulting solution was stirred until completely dissolved. Finally, purified water was added to a final volume of 200 ml, and the solution was mixed well.

TABLE 9
Ingredient contents of sample V-VII of 100 ml inhalation solution formulation

| Ingredients | Sample V | | Sample VI | | Sample VII | |
|---|---|---|---|---|---|---|
| Glycopyrrolate | 226 | mg | 226 | mg | 226 | mg |
| Olodaterol hydrochloride | 50 | mg | 50 | mg | 50 | mg |
| Edetate Disodium Dihydrate | 40 | | 40 | | 40 | |
| 50% benzalkonium chloride | 20 | mg | 20 | mg | 20 | mg |
| aqueous solution | | | | | | |
| Anhydrous citric acid | Adjust to pH 3.0 | | Adjust to pH 3.5 | | Adjust to pH 4.0 | |
| Purified water | 200 | ml | 200 | ml | 200 | ml |

The obtained solutions were filled into LDPE containers and sealed with aluminum foil, and stored at 40° C.±2° C./75%±5% RH. The stability data is shown in tables 10-12.

TABLE 10
The stability results of sample V (conditions: 40° C. ± 2° C./75% ± 5% RH)

| | Initial | 1 Month | 6 Months |
|---|---|---|---|
| Appearance | Colorless and clear | Colorless and clear | Colorless and clear |
| | solution | solution | solution |
| pH | 3.0 | 3.0 | 3.1 |
| Impurity GB-J | not detected | 0.11% | 0.54% |
| unknown maximum | 0.06% | not detected | not detected |
| impurity | | | |
| Total impurities | NA | 0.21% | 0.90% |
| Content of benzalkonium | 99.45% | 99.27% | 98.45% |
| chloride | | | |
| Content of edetate | 100.30% | 100.60% | 102.29% |
| disodium | | | |

NA: less than 0.05%

TABLE 11
The stability results of sample VI (conditions: 40° C. ± 2° C./75% ± 5% RH)

| | Initial | 1 Month | 6 Months |
|---|---|---|---|
| Appearance | Colorless and clear | Colorless and clear | Colorless and clear |
| | solution | solution | solution |
| pH | 3.5 | 3.6 | 3.6 |
| Impurity GB-J | not detected | 0.13% | 0.74% |
| unknown maximum | 0.1% | 0.13% | 0.74% |
| impurity | | | |
| Total impurities | 0.1% | 0.23% | 1.21% |
| Content of benzalkonium | 89.40% | 88.59% | 87.24% |
| chloride | | | |
| Content of edetate | 101.50% | 101.00% | 104.29% |
| disodium | | | |

TABLE 12
The stability results of sample VII (conditions: 40° C. ± 2° C./75% ± 5% RH)

| | Initial | 1 Month | 6 Months |
|---|---|---|---|
| Appearance | Colorless and clear | Colorless and clear | Colorless and clear |
| | solution | solution | solution |
| pH | 4.0 | 3.9 | 3.9 |
| Impurity GB-J | not detected | 0.20% | 1.19% |
| unknown maximum | 0.11% | 0.20% | 1.19% |
| impurity | | | |
| Total impurities | 0.11% | 0.30% | 1.76% |
| Content of benzalkonium | 125.15% | 122.81% | 120.47% |
| chloride | | | |
| Content of edetate | 100.10% | 99.90% | 99.24% |
| disodium | | | |

As can been seen from tables 10-12, at pH 3.0 GB and OH formulation solutions are most stable. Under the condition of pH 3-4, GB and OH formulation solutions are stable for 6 months at 40° C.±2° C./75%±5% RH.

While various embodiments of the present invention have been described above, it should be understood that they have been presented by way of example only, and not limitation. For example, the present invention is not limited to the physical arrangements or dimensions illustrated or described. Nor is the present invention limited to any particular design or materials of construction. As such, the breadth and scope of the present invention should not be limited to any of the above-described exemplary embodiments, but should be defined only in accordance with the following claims and their equivalents.

What is claimed is:

1. A liquid, propellant-free pharmaceutical preparation comprising: (a) glycopyrronium or a salt thereof (b) olodaterol or a salt thereof; (c) a solvent; (d) a pharmacologically acceptable stabilizer; and (e) a pharmacologically acceptable preservative.

2. The pharmaceutical preparation according to claim 1, wherein the pharmaceutical preparation has a pH in a range of from about 2.0 to about 7.5.

3. The pharmaceutical preparation according to claim 2, wherein the pharmaceutical preparation has a pH in a range of from about 3.0 to about 4.0.

4. The pharmaceutical preparation according to claim 1, wherein the olodaterol or a salt thereof is olodaterol hydrochloride and the glycopyrronium or a salt thereof is glycopyrrolate.

5. The pharmaceutical preparation according to claim 4, wherein the glycopyrrolate is present in an amount of from about 10 mg/100 ml to about 6 g/100 ml.

6. The pharmaceutical preparation according to claim 4, wherein the glycopyrrolate is present in an amount of from about 50 mg/100 ml to about 300 mg/100 ml.

7. The pharmaceutical preparation according to claim 4, wherein the olodaterol hydrochloride is present in an amount of from about 2.0 mg/100 ml to about 1.5 g/100 ml.

8. The pharmaceutical preparation according to claim 4, wherein the olodaterol hydrochloride is present in an amount of from about 10 mg/100 ml to about 50 mg/100 ml.

9. The pharmaceutical preparation according to claim 1, wherein the pharmacologically acceptable preservative is selected from the group consisting of benzalkonium chloride, benzoic acid, sodium benzoate, and combinations thereof.

10. The pharmaceutical preparation according to claim 9, wherein the preservative is present in an amount of from about 2 mg/100 ml to about 1000 mg/100 ml.

11. The pharmaceutical preparation according to claim 1, wherein the stabilizer is selected from the group consisting of edetic acid, edetate disodium dihydrate, edetate disodium, citric acid, and combinations thereof.

12. The pharmaceutical preparation according to claim 1, wherein the solvent is water.

13. The pharmaceutical preparation according to claim 11, wherein the stabilizer is present in an amount of from about 1 mg/100 ml to about 500 mg/100 ml.

14. The pharmaceutical preparation according to claim 1, wherein the pharmaceutical preparation further comprises a pharmacologically acceptable additive.

15. A method for administering the pharmaceutical preparation according to claim 1, comprising nebulizing a defined amount of the pharmaceutical preparation through a nozzle by the application of pressure to form an inhalable aerosol.

16. The method of claim 15, wherein the pharmaceutical preparation is nebulized using an inhaler device.

17. The method according to claim 15, wherein the defined amount of the pharmaceutical preparation is from about 5 to about 30 microliters.

18. The method according to claim 15, wherein the inhalable aerosol has a D50 less than about 8 μm.

19. The method according to claim 16, wherein the inhaler comprises a block function and a counter.

20. A method of treating asthma or COPD in a patient, comprising administering to the patient the pharmaceutical preparation the method of claim 15.

21. A method of treating asthma or COPD in a patient, comprising administering to the patient a pharmaceutical preparation of claim 1.
